(19) United States
(12) Patent Application Publication
(10) Pub. No.: US 2007/0021451 A1
(43) Pub. Date: Jan. 25, 2007

(54) Method for preventing or treating neurologic damage after spinal cord injury

(75) Inventors: Teruhisa Kazui, Hamamatsu-shi (JP); Naoki Washiyama, Hamamatsu-shi (JP); Enyi Shi, Shenyangshi (CN)

Correspondence Address: HAMRE, SCHUMANN, MUELLER & LARSON, P.C., P.O. BOX 2902, MINNEAPOLIS, MN, 55402-0902, US

(73) Assignees: HAMAMATSU UNIVERSITY SCHOOL OF MEDICINE, Hamamatsu-shi (JP); NIPPON SHINYAKU CO., LTD., Kyoto-shi (JP)

(21) Appl. No.: 11/335,422

(22) Filed: Jan. 19, 2006

(30) Foreign Application Priority Data
Jul. 20, 2005 (JP) 2005-210368

(51) Int. Cl. A61K 31/513

(52) U.S. Cl. 514/269

(57) ABSTRACT

The present invention provides a method for preventing and/or treating neurologic damage after spinal cord injury in a mammal including human in need thereof which comprises administering an effective amount of a compound of the general formula [I]: 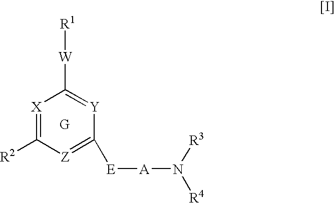 or a salt, or a solvate thereof, and a use thereof.

### TECHNICAL FIELD

The present invention is related to a method for preventing and/or treating neurologic damage after spinal cord injury.

### BACKGROUND ART

The spinal cord is a long cylindrical tissue connected to medulla oblongata, which passes down through the spinal canal and plays a role in the neurotransmission between brain and spinal nerve. The spinal cord is comprised of central canal, H-shaped gray matter, and white matter from the center to the outer layer. The anterior portion of H-shaped gray matter (anterior horn) is built up with motor neurons, which neurons transmit information from brain or spinal cord to muscle, and thereby stimulating movement. The posterior portion of H-shaped gray matter (posterior horn) is built up with sensory neurons, which transmit sensory information from other parts of the body through the spinal cord to brain. The surrounding white matter contains columns of nerve fibers that carry sensory information from the rest of the body to the brain (ascending tracts) and columns that carry impulses from the brain to the muscles (descending tracts). In human, 31 pairs of spinal cord nerve play an important role in the transmission between the brain and other parts of the body. Thus, if spinal cord is injured and neurons are damaged by traffic accident, spinal cord disease such as spinal cord deformation, tumor, ischemia due to blockage of blood flow or the like, motor disorders such as motor paralysis of limbs and apraxia, or anesthesia may occur.

The blood flow into spinal cord, especially into the anterior spinal cord is mainly supplied via the branch vessels of the aorta, and hence the interruption of blood flow in any branch vessel can result in spinal cord ischemia. Blockage of blood flow may be caused by severe atherosclerosis, aorta dissecting, thrombus etc., and also purposely during aortic aneurysmectomy. Recently, the risk of ischemic spinal cord injury tends to become high as the increase of arteriosclerotic diseases in accordance with the aging of society and the change of dietary pattern.

Of arteriosclerotic diseases, aortic aneurysm is usually silent until it bursts. However, once it bursts, severe symptoms which often lead to death develop. Accordingly, appropriate treatment must be applied before burst. Treatment can be performed medically and/or surgically depending on the location and size of aneurysm, conditions of patient, and the like. When an aneurysm reaches to a given size, surgical treatment is inevitable. Surgical treatment generally involves occlusion of aorta in the vicinity of the aortic aneurysm, excision of aneurysm and replacement with artificial vessel (“surgical repair of aneurysm”).

The surgical repair of descending thoracic aortic aneurysm or thoracoabdominal aortic aneurysm among aortic aneurysms requires interrupting the blood flow around descending aorta region which includes a site that is important for the blood supply to the spinal cord. Therefore, this operation may cause a spinal cord injury due to the spinal cord ischemia during or after operation, which possibly results in severe complications such as paraplegia and rectovesical impairment. In fact, it has been reported that the incidence of paraplegia reaches about 10% in the surgical repair of descending thoracic aortic aneurysm or thoracoabdominal aortic aneurysm. The spinal cord ischemia-related complications of surgical repair of aortic aneurysm, when milder ones are took into consideration, would exert grave influences on a patient or society.

The mechanism of spinal cord injury during operations on the descending thoracic aortic aneurysm or thoracoabdominal aortic aneurysm is believed to be related primarily to tissue ischemia directly. Numerous surgical techniques and pharmacologic interventions have been used to protect spinal cords, but the complication still cannot be prevented completely (Svensson L. G., et. al., J. Vasc. Surg., 1993; 17:357-70, Gharagozloo F., et. al., Chest., 1996; 109: 799-809).

Although the precise neurochemical sequels after ischemia remain to be clarified, the action of voltage-sensitive Na⁺ channels (VSSCs), as well as Ca²⁺ channels, is considered to be involved in the pathogenesis of ischemic neurologic injury (Barone F. C. et. al., Stroke. 1995, 26: 1683-90, Lipton P., Physiol Rev. 1999, 79:1431-568). The opening of VSSCs facilitates ischemic glutamate release and intercellular Ca²⁺ overload (Koch R. A. et. al., J Neurosci. 1994; 14:2585-93; Kanai Y. et. al., Trends Neurosci. 1993; 16: 365-70), which have been considered to be the major cause of neuronal degeneration after cerebral ischemia (Choi D. W. et. al., J Neurosci. 1988; 8: 185-96; Oka M. et. al., Life Sci. 2000, 67: 2331-43). Consistent with these findings, Na⁺ and Ca²⁺ channel blockers (Na⁺/Ca²⁺ channel blockers) have been proved to be neuroprotective in models of spinal cord and cerebral ischemia (Gangemi J. J. et. al., Ann Thorac Surg. 2000; 69: 1744-9; Gemba T. et. al., J Pharmacol Exp Ther. 1993; 265: 463-7). However, the Na⁺/Ca²⁺ channel blockers have been reported to have severe cardiovascular side effects such as hypertension and arrhythmia, which restricted the clinical applicability of their protective effects on the cerebrospinal nervous system (Tanaka K. et. al., Brain Res. 2002; 924: 98-108).

As described above, when the spinal cord is injured and neurons are damaged, the risk of onset of paraplegia, rectovesical impairment or the like becomes high, and therefore researches have been focused on the development of a method for preventing and/or treating neuronal damage at the time of spinal cord ischemia. For example, 2-amino-6-trifluoromethoxybenzothiazole (Riluzole™) was reported to be effective in preventing ischemic spinal cord injury induced by aortic cross-clamping (WO 00/66121). Also, the effects of pyrazolone compounds are suggested in the protection and/or treatment of damage due to ischemic spinal cord injury in model animals of spinal cord injury after surgical repair of aneurysm (JP-2004-67585, A).

However, the ischemic spinal cord injury due to an accident or surgery brings about such a significant impact on patients and society that it is still strongly required to develop novel pharmaceuticals which exhibit excellent neuronal protecting effects under spinal cord ischemic condition.

### DISCLOSURE OF INVENTION

The present invention provides a method for preventing and/or treating neuronal damage due to spinal cord injury.

The present inventors have found that certain compounds among those known as a Na⁺/Ca²⁺ channel blocker are useful in the prevention and/or the treatment of neuronal damage due to spinal cord injury. Thus, the present invention provides a method for preventing or treating neurologic damage after spinal cord injury in a mammal including human in need thereof, which comprises administering an effective amount of a compound of the following general formula [I]: 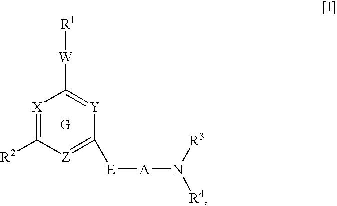
or a salt thereof, or a solvate thereof, as an active ingredient.

In the formula [I], R¹ represents an aryl group that may be substituted or a 5- through 10-membered heteroaromatic group that may be substituted, wherein the heteroaromatic group may be a monocyclic or fused ring system containing at least one heteroatom selected from the group consisting of nitrogen, oxygen, and sulfur as a ring member. Additionally, the aryl and heteroaromatic group each may be substituted with one to three substituents which are the same or different and selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro.

R² represents hydrogen, alkyl, alkenyl, cycloalkyl, cycloalkylalkyl, hydroxyalkyl, haloalkyl, alkoxy, alkylthio, amino, monoalkylamino, dialkylamino, or phenyl. The phenyl may be substituted with one to three substituents which are the same or different and selected from the group consisting of halogen, alkyl, and alkoxy.

R³ and R⁴ may be the same or different and each represents hydrogen or alkyl that may be substituted by one or two substituents which are the same or different and selected from the group consisting of hydroxy, alkoxy, amino, monoalkylamino, and dialkylamino, or R³ and R⁴ taken together with the adjacent N atom may represent a 4- through 8-membered cyclic amino group of the formula NR³R⁴. The cyclic amino group may have N, O, or S in addition to said N atom as a ring member and be substituted with one to three substituents that are the same or different and selected from the group consisting of alkyl, alkoxy, hydroxy, oxo, amino, monoalkylamino, dialkylamino, aryl that may be substituted, and pyridyl that may be substituted.

In addition, R³ and R⁴ taken together with the adjacent N atom may form an oxide.

A represents alkylene of 2-10 carbon atoms which may be substituted by alkoxy, hydroxy, or oxo in optional substitutable positions.

E represents O or S.

W represents a single bond, O, S, or (CH₂)ₙ, wherein CH₂ may be substituted with alkyl and n is an integer of 1 or 2.

X, Y, and Z may be the same or different and each represents CH, CR or N, wherein R represents alkyl, with the proviso that the case in which X, Y, and Z concurrently represent CH or CR wherein R represents alkyl is excluded.

Ring G represents pyridine, pyrimidine, or 1,3,5-triazine.

When one to three of X, Y, and Z represent N, one of them may form an oxide.

### BRIEF DESCRIPTION OF DRAWINGS

FIG. 1 is representative photographs of the lumbar spinal cord sections stained with TTC. The specimens from the sham group (A), from the control group (B), from rabbits that received NS-7 before ischemia (C) and from rabbits that received NS-7 at the onset of reperfusion (D) are shown. Viable tissue is brick red, whereas infarcted tissue is pale. White matter is also pale.

FIG. 2 is a graph showing the area of infarction at mid lumbar that is calculated based on FIG. 1 with National Institutes of Health Image software, which is expressed as a percentage of the area of the whole gray matter. *P<0.05 compared with the control group.

FIG. 3 is a photograph showing the specimen of lumbar stained with HE. A, sham-operated group; B, control group; C, treated group that received NS-7 before ischemia; and D, treated group that received NS-7 at the onset of reperfusion. Arrows exhibits eosinophilic neuronal degeneration.

FIG. 4 is a graph showing mean histologic scores of ischemic injury at different sites of the spinal cord. LT, Low thoracic; UL, upper lumbar; ML, mid lumbar; LL, low lumbar. *P<0.05, #P<0.01 compared with the control group (Mann-Whitney U tests).

FIG. 5 is a photograph showing the TUNEL-stained lumbar slice (200×). A, sham-operated group; B, control group; C, treated group that received NS-7 before ischemia; and D, treated group that received NS-7 at the onset of reperfusion. Arrows exhibits TUNEL-positive motor neuron.

FIG. 6 is a graph showing the number of motor neurons that were positive in TUNEL staining (the values from 3 sections were averaged). *P<0.05 compared with the control group.

### BEST MODE FOR CARRYING OUT THE INVENTION

The compounds of the formula [I], an active ingredient used in the method of the present invention, are known compounds and reported to exhibit an excellent neuronal death inhibitory action in the acute phase of a cerebrovascular disease (WO/96/07641). It is also known that excitatory amino acids participate in the neuronal injury and the receptors of such amino acids include N-methyl-D-aspartate (hereinafter referred to as NMDA) receptors and those different from NMDA receptors (hereinafter referred to as “non-NMDA receptors”). It has been confirmed that the compound [I] used in the present method does not act on the NMDA receptors (WO/96/07641).

However, it has not been known whether the compound [I] has neuroprotective effects under spinal cord ischemic condition, and is useful as a pharmaceutical agent for the prevention and/or treatment of neuronal damage due to ischemic spinal cord injury

The compounds of the formula [I], an active ingredient used in the method of the present invention, include those shown in (i) to (iv) below.

(i) The compounds (I) wherein NR³R⁴ is a 4- through 8-membered cyclic amino group; and A is an alkylene group of 4-10 carbon atoms. The cyclic amino group may have oxygen or sulfur as a ring member and may have alkyl, alkoxy, hydroxy, oxo, amino, monoalkylamino, dialkylamino, pyridyl, or aryl as a substituent(s). The aryl may be substituted with one to three substituents which are the same or different and selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro.

(ii) The compounds (I) wherein R¹ is a 5- through 10-membered heteroaromatic group; R² is hydrogen, A is an alkylene group of 2-3 carbon atoms, which may be substituted with alkoxy, hydroxy, or oxo in an optional substitutable position; and E is O. The heteroaromatic group is a monocyclic or fused ring system containing at least one heteroatom selected from the group consisting of nitrogen, oxygen, and sulfur as a ring constituent atom and may be substituted by one to three substituents which are the same or different and selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro.

(iii) The compounds (I) wherein R¹ is a 5- through 10-membered heteroaromatic group which may be a monocyclic or fused ring system containing at least one heteroatom selected from the group consisting of nitrogen, oxygen, and sulfur as a ring constituent atom, said heteroaromatic group being optionally substituted by 1-3 same or different substituents selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro; R² is alkyl, alkenyl, cycloalkyl, cycloalkylalkyl, hydroxyalkyl, haloalkyl, alkoxy, alkylthio, amino, monoalkylamino, dialkylamino, or phenyl, said phenyl being optionally substituted by one to three substituents which are the same or different and selected from the group consisting of halogen, alkyl, and alkoxy; and A is an alkylene group of 2-3 carbon atoms, which may be substituted by alkoxy, hydroxy, or oxo in an optional substitutable position.

(iv) The compounds (I) wherein R¹ is a 5- through 10-membered heteroaromatic group which may be a monocyclic or fused ring system and contain at least one heteroatom selected from the group consisting of nitrogen, oxygen and sulfur as a ring constituent atom and which may be substituted by one to three substituents which are the same or different and selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro; NR³R⁴ is piperazino which may be unsubstituted or substituted by alkyl, alkoxy, hydroxy, oxo, amino, monoalkylamino, dialkylamino, pyridyl, or aryl, said aryl being optionally substituted by one to three substituents which are the same or different and selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro.

As used throughout this specification, the alkyl means a straight-chain or branched alkyl group of 1-6 carbon atoms, such as methyl, ethyl, n-propyl, isopropyl, n-butyl, isobutyl, sec-butyl, tert-butyl, n-pentyl, isopentyl, n-hexyl, and isohexyl. Particularly preferred is an alkyl group of 1-4 carbon atoms.

The alkenyl means a group of 2-6 carbon atoms, such as vinyl, allyl, 3-butenyl, 2-pentenyl, and 4-hexenyl.

The cycloalkyl is preferably a group of 3-10 carbon atoms, such as cyclopropyl, cyclobutyl, cyclopentyl, cyclohexyl, cycloheptyl, cyclooctyl, 1-adamantyl, and 2-adamantyl.

The aryl means a group of 6-13 carbon atoms, such as phenyl, 1-naphthyl, 2-naphthyl, or biphenyl. Particularly preferred is phenyl.

The aralkyl means a group of 7-13 carbon atoms, whose alkyl moiety is either straight-chain or branched, thus including benzyl, phenethyl, phenylpropyl, phenylbutyl, diphenylmethyl, and naphthylmethyl, among others.

The halogen includes chlorine, fluorine, bromine, and iodine.

The alkoxy is preferably a straight-chain or branched group of 1-6 carbon atoms, such as methoxy, ethoxy, n-propoxy, isopropoxy, n-butoxy, isobutoxy, sec-butoxy, tert-butoxy, n-pentyloxy, isopentyloxy, n-hexyloxy, and isohexyloxy.

The alkanoyl means a straight-chain or branched group of 1-6 carbon atoms, such as acetyl, propanoyl, butanoyl, isobutanoyl, pentanoyl, hexanoyl, and 2-methylpentanoyl.

The alkylthio is preferably a group having a straight-chain or branched alkyl moiety of 1-6 carbon atoms, such as methylthio, ethylthio, n-propylthio, isopropylthio, n-butylthio, isobutylthio, sec-butylthio, tert-butylthio, n-pentylthio, isopentylthio, n-hexylthio, and isohexylthio.

The alkylsulfonyl is preferably a group having a straight-chain or branched alkyl moiety of 1-6 carbon atoms, such as methylsulfonyl, ethylsulfonyl, n-propylsulfonyl, isopropylsulfonyl, n-butylsulfonyl, isobutylsulfonyl, sec-butylsulfonyl, tert-butylsulfonyl, n-pentylsulfonyl, isopentylsulfonyl, n-hexylsulfonyl, or isohexylsulfonyl.

The hydroxyalkyl is a group having a straight-chain or branched alkyl moiety of 1-6 carbon atoms, such as 2-hydroxyethyl, 3-hydroxypropyl, 2-hydroxypropyl, 4-hydroxybutyl, 3-hydroxybutyl, 5-hydroxypentyl, and 6-hydroxyhexyl.

The haloalkyl is a group having a straight-chain or branched alkyl moiety of 1-6 carbon atoms, such as trifluoromethyl, fluoromethyl, 2-bromoethyl, and 3-chloroethyl.

The monoalkylamino is a group having a straight-chain or branched alkyl moiety of 1-6 carbon atoms, such as methylamino, ethylamino, propylamino, butylamino, heptylamino, and hexylamino.

The dialkylamino is a group having straight-chain or branched alkyl moieties of 1-6 carbon atoms, such as dimethylamino, diethylamino, dipropylamino, dibutylamino, diheptylamino, and dihexylamino.

The alkoxycarbonyl is preferably a straight-chain or branched group of 2-7 carbon atoms, such as methoxycarbonyl, ethoxycarbonyl, n-propoxycarbonyl, isopropoxycarbonyl, n-butoxycarbonyl, isobutoxycarbonyl, sec-butoxycarbonyl, tert-butoxycarbonyl, n-pentyloxycarbonyl, isopentyloxycarbonyl, n-hexyloxycarbonyl, and isohexyloxycarbonyl.

The cycloalkyloxy is preferably a group of 3-10 carbon atoms, such as cyclopropyloxy, cyclobutyloxy, cyclopentyloxy, cyclohexyloxy, cycloheptyloxy, cyclooctyloxy, and 2-adamantyloxy.

The cycloalkylalkyl is preferably a group of 4-11 carbon atoms, such as cyclopropylmethyl, cyclopropylethyl, cyclobutylmethyl, cyclobutylethyl, cyclopentylmethyl, cyclopentylethyl, cyclopentylpropyl, cyclohexylmethyl, cyclohexylethyl, cyclohexylpropyl, cycloheptylmethyl, and 2-adamantylmethyl.

The 4- through 8-membered cyclic amino group includes azetidin-1-yl, pyrrolidin-1-yl, piperidino, hexamethylenimino, tetrahydropyridino, octahydroazocin-1-yl, piperazin-1-yl, homopiperazin-1-yl, morpholino, and thiomorpholino.

The substituent that may be present on said cyclic amino group includes alkyl, alkoxy, hydroxy, oxo, amino, monoalkylamino, dialkylamino, aryl that may be substituted, and pyridyl that may be substituted. The substituent that may be present on the aryl or pyridyl includes the groups mentioned for the substituent on R¹.

The 5- through 10-membered heteroaromatic group is a monocyclic or fused ring system, which contains at least 1 heteroatom selected from the group consisting of oxygen, sulfur and nitrogen. Thus, for example, 2-pyridyl, 3-pyridyl, 4-pyridyl, 2-thienyl, 2-furyl, 2-pyrimidinyl, 4-pyrimidinyl, 5-pyrimidinyl, 3-pyridazinyl, 4-pyridazinyl, 1-isoquinolyl, 4-isoquinolyl, 2-quinazolinyl and 1-methyl-2-indolyl can be mentioned.

The alkylene represented by A may be straight-chain or branched. For the purpose of using as a therapeutic agent for protecting nerves from the spinal cord ischemia and preventing and/or treating neuronal damage, A is preferably an alkylene group of 3-6 carbon atoms, more preferably a group of 4-6 carbon atoms.

E preferably represents O.

W preferably represents a single bond.

X, Y, and Z are preferably such that X=Z=N with Y═CH, or Z=N with X═Y═CH. The former combination is particularly preferred.

R¹ preferably represents halogen-substituted phenyl, and particularly fluorophenyl.

R² is preferably alkyl or haloalkyl, more preferably alkyl, and far more preferably methyl.

Preferably, R³ and R⁴ taken together with the adjacent N atom represent a cyclic amino group of the formula —NR³R⁴. In particular, a cyclic amino group containing only one nitrogen atom as a ring-constituent heteroatom is preferred. Especially preferred is piperidino.

Example of preferred compounds includes those represented by the general formula [Ia]:

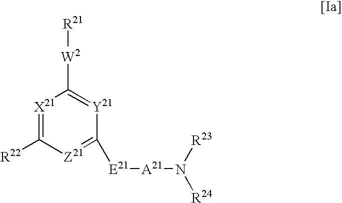

In the formula [Ia], A²¹ represents alkylene of 4-6 carbon atoms.

E21 represents O.

X²¹=Z²¹=N with Y²¹═CH, or X²¹═Y²¹═CH with Z²¹=N.

R²¹ represents halogen-substituted phenyl.

R²² represents alkyl or haloalkyl.

R²³ and R²⁴ taken together with the adjacent N atom represent a 4 through 8 membered cyclic amino group of the formula —NR²³R²⁴, said cyclic amino group containing only one nitrogen atom as a ring constituent heteroatom.

W² represents a single bond.

More preferred compounds include:
4-(4-fluorophenyl)-2-methyl-6-(4-piperidinobutoxy)pyrimidine,
4-(4-fluorophenyl)-2-methyl-6-(1-methyl-4-piperidinobutoxy)pyrimidine,
4-(4-fluorophenyl)-2-methyl-6-(5-piperidinopentyloxy)pyrimidine,
4-(4-fluorophenyl)-2-methyl-6-(6-piperidinohexyloxy)pyrimidine,
2-(4-fluorophenyl)-4-methyl-6-(4-piperidinobutoxy)pyrimidine,
4-(4-fluorophenyl)-2-methyl-6-(3-piperidinopropoxy)pyridine, and
4-(4-fluorophenyl)-2-methyl-6-(5-piperidinopentyloxy)pyridine,
   or a salt thereof.

The most preferred compound is 4-(4-fluorophenyl)-2-methyl-6-(5-piperidinopentyloxy)pyridine and a salt thereof.

The compound used as an active ingredient in the method of the present invention is preferentially distributed to the membrane-enriched synaptosomal fraction of brain rather than the heart and has a much higher affinity for brain VSSCs compared to cardiac VSSCs, indicating that it might exert less side effects on cardiac function (Shimidzu T. et. al., Naunyn Schmiedebergs Arch. Pharmacol. 1997; 355: 601-8).

The solvate of compound [I] useful as an active ingredient in the method of the present invention includes hydrate and ethanolate.

The salts of compound [I] include those formed with an inorganic acid such as hydrochloric acid, sulfuric acid, nitric acid, phosphoric acid, hydrofluoric acid and hydrobromic acid, or those with organic acid such as acetic acid, tartaric acid, lactic acid, citric acid, fumaric acid, maleic acid, succinic acid, methanesulfonic acid, ethanesulfonic acid, benzenesulfonic acid, toluenesulfonic acid, naphthalenesulfonic acid and camphorsulfonic acid.

While some species of the compound [I] contain asymmetric carbon, the respective optical isomers as well as the racemic mixtures are also useful for the present method. Compound [I] may show crystal polymorphism. The polymorphs in such cases are also useful for the present invention.

The compound [I], a salt or a solvate thereof which is used in the method of the present invention can be prepared and purified, if necessary, by known procedures such as those described in WO 96/07641 and WO 02/20492.

A pharmaceutical composition comprising a compound of the formula [I], a salt or a solvate thereof as an active ingredient is useful for protecting neuronal cells against spinal cord ischemia, and preventing and/or treating neuronal damage (injury). Accordingly, the present invention also includes a method for protecting spinal cord neuronal cells against spinal cord ischemia in a mammal including human in need thereof which comprises administering an effective amount of a compound of the formula [I], or a salt thereof, or a solvate thereof.

The term “protecting neuronal cells against a spinal cord ischemia”, as used herein, means preventing spinal cord neuronal injury under the condition where blood flow into spinal cord is interrupted or restricted, or promoting restoration when the cells are injured.

In another aspect, the present invention relates to use of a compound of the formula [I], or a salt or a solvate thereof in the manufacture of a pharmaceutical composition comprising the compound.

The dosage of the present pharmaceutical composition is preferably established with reference to the patient's conditions such as age, body weight, and the route of administration, and the like; however, in general, the daily dosage of a compound of the formula [I] as an active ingredient for human adults may range from 0.1 mg/patient to 1 g/patient and preferably from 1 mg/patient to 300 mg/patient for oral administration. In the case of parenteral administration, the daily dosage in general may range from 0.01 mg/patient to 100 mg/patient and preferably from 0.1 mg/patient to 30 mg/patient. A lower or higher dosage may be needed in some cases. The above-mentioned dosage can be preferably administered in 2-4 divided doses.

For use as a medicine, a compound of the formula [I], a salt or a solvate thereof as an active ingredient can be administered as it is; however, it is generally preferred to be administered after formulating into a pharmaceutical composition containing an active ingredient in a pharmaceutically acceptable nontoxic, inert carrier. The amount of an active ingredient in a pharmaceutical composition varies depending on the form but is generally from 0.01% to 99.5% by weight, preferably from 0.5% to 90% by weight.

As the carrier, one or more of solid, semi-solid, or liquid diluent, filler, and other auxiliaries for formulations can be used. The pharmaceutical composition for carrying out the present invention is preferably administered in unit dosage forms. The pharmaceutical composition of the present invention can be administered orally, parenterally (e.g. intravenously), locally (e.g. transdermally), or rectally. Intravenous or oral administration is preferred, and intravenous administration is particularly preferred. The pharmaceutical composition can be prepared by any methods known in the art, preferably, the one described in WO 96/07641.

Oral administration can be carried out using solid or liquid unit dosage forms such as bulk powders, powders, tablets, dragees, capsules, granules, suspensions, solutions, syrups, drops, and sublingual tablets.

Bulk powders can be manufactured by comminuting an active substance into a finely-divided form. Powders can be manufactured by comminuting the active substance into a finely-divided form and blending it with a similarly comminuted pharmaceutical carrier, for example, an edible carbohydrate such as starch or mannitol. Where necessary, a corrigent, a preservative, a dispersant, a coloring agent, a perfume, or the like can also be added.

Capsules can be manufactured by filling said finely-divided bulk powders or powders comminuted as described above, or granules described below for tablets, in capsule shells such as gelatin capsule shells. Preceding the filling operation, a lubricant or a fluidizing agent, such as colloidal silica, talc, magnesium stearate, calcium stearate and solid polyethylene glycol, can be blended with the powders. Improvement in the efficacy of the drug after ingestion can be expected when a disintegrator or a solubilizer, such as carboxymethylcellulose, carboxymethylcellulose calcium, low-substitution-degree hydroxypropylcellulose, croscarmellose sodium, sodium carboxymethylstarch, calcium carbonate and sodium carbonate, is added.

Soft capsules can be provided by suspending said finely-divided powders of compound [I] in vegetable oil, polyethylene glycol, glycerin, or a surfactant and wrapping the suspension in gelatin sheets. Tablets can be manufactured by adding an excipient to said powders, granulating or slugging the mixture, adding a disintegrator and/or a lubricant, and compressing the whole composition. A powdery mixture can be prepared by mixing said finely-divided powders with said diluent or a base. Where necessary, a binder (e.g. carboxymethylcellulose sodium, methylcellulose, hydroxypropylmethylcellulose, gelatin, polyvinylpyrrolidone, polyvinyl alcohol, etc.), a dissolution retardant (e.g. paraffin), a reabsorption agent (e.g. quaternary salts), and an adsorbent (e.g. bentonite, kaolin, dicalcium phosphate, etc.) can be added.

The powdery mixture can be processed into granules by wetting it with a binder, for example, syrup, starch paste, gum arabic, a solution of cellulose, or a solution of a high polymer, stirring to mix, drying it, and pulverizing the same. Instead of granulating as above, it is possible to compress the powders with a tablet machine and crush the resulting slugs of crude form to prepare granules. The resulting granules can be protected against interadhesion by the addition of a lubricant such as stearic acid, a salt of stearic acid, talc and mineral oil. The mixture thus lubricated is then compressed. The resulting uncoated tablets can be coated with a film coating composition or a sugar coating composition.

The compound [I] can be mixed with a free-flowing inert carrier and the mixture be directly compressed without resorting to the above-mentioned granulation or slugging process. A transparent or translucent protective coat comprising a hermetic shellac coat, a sugar or polymer coat, or a polishing wax coat can also be applied. Other forms for oral administration such as a solution, a syrup, and an elixir can also be provided in unit dosage forms each containing a predetermined amount of the compound [I]. Syrups can be manufactured by dissolving the compound [I] in suitable flavored aqueous media, while elixirs can be manufactured using nontoxic alcoholic vehicles. Suspensions can be formulated by dispersing the compound [I] in nontoxic vehicles. Where necessary, solubilizers or emulsifiers (e.g. ethoxylated isostearyl alcohol, polyoxyethylene sorbitol ester, etc.), preservatives, flavorants (e.g. peppermint oil, saccharin, etc.), or the like can also be added.

Where necessary, the unit dosage formulation for oral administration can be microencapsulated. This formulation can be coated or embedded in a polymer, wax or the like, to provide a prolonged action or sustained release dosage form.

Parenteral administration can be carried out using injection, drip infusion or sustained arterial or intravenous infusion. Injection can be carried out using liquid dosage forms for subcutaneous, intramuscular, or intravenous injection, e.g. solutions and suspensions.

The pharmaceutical composition for injection, drip infusion or sustained arterial or intravenous infusion can be manufactured by suspending or dissolving the compound of interest in an injectable nontoxic liquid vehicle, for example an aqueous vehicle or an oily vehicle, and sterilizing the resulting suspension or solution. For isotonizing an injection, a nontoxic salt or salt solution can be added. Moreover, stabilizers, preservatives, emulsifiers, or the like may also be added.

Rectal administration can be carried out using suppositories manufactured by dissolving or suspending the compound in a low-melting water-soluble or water-insoluble solid carrier such as polyethylene glycol, cacao butter, semisynthetic oil (e.g. Witepsol™), a higher ester (e.g. myristyl palmitate) and a mixture thereof. A formulation for oral administration can be a solid or liquid unit dosage forms such as bulk powders, powders, tablets, dragees, capsules, granules, suspensions, solutions, syrups, drops, sublingual tablets and other suitable dosage forms and can be prepared by using additives, for example, excipient, such as glucose, lactose, D-mannitol, starch and crystalline cellulose; disintegrant or disintegrant aids, such as carboxymethylcellulose, starch or carboxymethylcellulose calcium; binder, such as hydroxypropylcellulose, hydroxypropylmethylcellulose, polyvinylpyrrolidone and gelatine; lubricant, such as magnesium stearate and talc; coating agent, such as hydroxypropylmethylcellulose, sucrose, polyethylene glycol and titanium oxide; base, such as petrolatum, liquid paraffin, polyethylene glycol, gelatin, kaolin, glycerin, purified water and hard fat.

The pharmaceutical composition for carrying out the present invention is effective for the prevention and/or treatment (including alleviation) of neurologic damage after spinal cord injury. In particular, the pharmaceutical composition for carrying out the present invention is useful as a therapeutic agent for protecting spinal cord nerve cells and prevents or attenuates the progression or development of cell damage. It is especially useful for improving the conditions of the injured cells or even restoring them to normal condition after the spinal cord has been exposed to damage. In particular, this pharmaceutical composition is useful for protecting or treating neurons after ischemic spinal cord injury.

The term “spinal cord injury” as used herein means any kind of damage of the spinal cord neuronal cells. This definition includes that necrosis or apoptosis of spinal cord neurons progresses after ischemia whereby the neurotransmission via the injured part of spinal cord is impaired to result in motor disturbance or sensory loss.

When the pharmaceutical composition is used for preventing ischemic spinal cord invasion (injury) related to the surgical repair of aortic aneurysm, it can be administered prophylactically. Prophylactic administration may be performed either orally or parenterally. The parenteral administration including injection, drip infusion or sustained arterial or intravenous infusion may be performed prophylactically before, during or after operation. Particularly, in the operation of the descending thoracic aortic aneurysm or thoracoabdominal aortic aneurysm, it is preferred that the pharmaceutical composition is administrated several times preceding to, during, or preferably at the onset of reperfusion of aorta blood flow. Additionally, the pharmaceutical composition can be administrated parenterally (e.g., intravenously or intra-arterially) or orally to a patient having neurologic injury after spinal cord ischemia in order to prevent or alleviate the deterioration of disease.

As described above, the pharmaceutical composition for carrying out the present invention is effective irrespective of the time of administration including before or during the occurrence of spinal cord ischemia, or at the onset of reperfusion, and hence is suitable for the prevention and/or treatment of neurologic injury in the case not only where the ischemic spinal cord invasion can be expected (e.g., in the case of surgical repair of aortic aneurysm) but also where the spinal cord ischemia cannot be expected (e.g., in the case of accident or attack). The pharmaceutical composition is also useful in the prevention and/or treatment of neurologic injury after spinal cord ischemia caused by an accident or attack. For example, the pharmaceutical composition can be administered as first-aid treatment in order to inhibit the progress of neuronal damage and/or promote the restoration and thereby inhibiting or alleviating the possible subsequent complications.

### EXAMPLES

The present invention is further illustrated by the following examples, but should not be construed as being limited by the same.

### Preparation Example 1

### 2-(4-Fluorophenyl)-4-(4-piperidinobutoxy)-6-methylpyridine hydrochloride

### (1) 4-(4-Chlorobutoxy)-2-(4-fluorophenyl)-6-methylpyridine

A mixture of 2.5 g of 2-(4-fluorophenyl)-4-hydroxy-6-methylpyridine, 3.16 g of 1-bromo-4-chlorobutane, 1.7 g of silver carbonate, and 100 ml of toluene was heated to reflux for 40 hours. This reaction mixture was filtered to remove insolubles and the filtrate was concentrated. The residue was purified with silica gel column chromatography to provide 1.45 g of the title compound as white crystals. M.p. 59-61° C.

### (2) 2-(4-Fluorophenyl)-4-(4-piperidinobutoxy)-6-methylpyridine hydrochloride

A mixture of 1.45 g of 4-(4-chlorobutoxy)-2-(4-fluorophenyl)-6-methylpyridine obtained in (1) above, 1.26 g of piperidine, and 12 ml of DMF was stirred at 100° C. for 1.5 hours. This reaction mixture was cooled, poured into iced water, and extracted with ethyl acetate. The organic layer was washed with brine several times, dried over MgSO₄, and then concentrated. The residue was purified with silica gel column chromatography to provide 1.2 g of the objective compound as oil. This oil was dissolved in methanol and the solution was adjusted to pH 5 with 3.5 ml of 1N hydrochloric acid and concentrated. To the residue was added ether and the resulting crystal crop was collected by filtration and recrystallized from the mixture of acetonitrile and ether to provide 1.02 g of the title compound as white crystals. M.p. 164-166° C.

Elemental analysis for C₂₁H₂₇FN₂O.HCl:

Calcd. (%): C, 66.57; H, 7.45; N, 7.39.

Found. (%): C, 66.21; H, 7.45; N, 7.09.

In the same manner as Preparation Example 1, the following compounds were synthesized.

### Preparation Example 2

### 4-(4-Fluorophenyl)-2-methyl-6-(5-piperidinopentyloxy)pyridine hydrochloride (hereinafter, referred to as “NS-7”)

M.p. 138-140° C.

Elemental analysis for C₂₂H₂₉FN₂O.HCl:

Calcd. (%): C, 67.25; H, 7.70; N, 7.13.

Found. (%): C, 67.00; H, 7.68; N, 6.95.

### Preparation Example 3

### 4-(4-Fluorophenyl)-2-methyl-6-(5-piperidinopentyloxy)pyrimidine

### (1) 4-(4-Fluorophenyl)-6-hydroxy-2-methylpyrimidine

A mixture of 8.7 kg of ethyl 3-(4-fluorophenyl)-3-oxopropionate, 11.7 kg of acetamidine hydrochloride, 28.6 g of potassium carbonate, and 34.5 L of methanol was stirred at 50° C. for 5 hours. The reaction mixture was filtered to remove insolubles and the filtrate was washed with methanol. The mother liquid and washes were combined with water and neutralized with 18% hydrochloric acid. After neutralization, the mixture solution was heated to reflux for 4 hours for the aging of crystal. The resulting crystals were isolated by centrifugation, washed with water and dried to give 7.4 kg of the title compound as white crystals.

### (2) 4-Chloro-6-(4-fluorophenyl)-2-methylpyridine

A mixture of 13.8 kg of 4-(4-fluorophenyl)-6-hydroxy-2-methylpyrimidine and 11.5 kg of phosphorus oxychloride in acetonitrile was heated to reflux and stirred for 4 hours. Water was added to the reaction solution. The precipitated crystals were isolated, washed with water and dried to give 14.6 kg of the title compound as pale yellow crystalline.

### (3) 4-(4-Fluorophenyl)-2-methyl-6-(5-piperidinopentyloxy)pyrimidine

A mixture of 13.8 kg of 4-chloro-6-(4-fluorophenyl)-2-methylpyridine, 4.8 kg of 60% sodium hydride, 130 kg of cyclohexane, and 10.6 kg of 5-piperidino-1-pentanol was heated to reflux for 4 hours.

### Preparation Example 4

### 4-(4-Fluorophenyl)-2-methyl-6-(5-piperidinopentyloxy)pyrimidine hemihydrate

The reaction solution obtained in the Preparation Example 3 was cooled and mixed with 55 kg of water, and stirred at room temperature for 1 hour. The aqueous layer was removed. The organic layer was extracted with 3% hydrochloric acid and neutralized with 4% aqueous sodium hydroxide. The precipitated crystals were isolated. The mixture of the resulting crystals and 120 kg of acetone was treated with activated charcoals and the insolubles were removed by filtration. To the filtrates was added 85.2 kg of water and the crystals precipitated. The precipitated crystals were isolated and dried to give 19.2 kg of the title compound as white crystals (yield: 87%). M.p. 62.0-63.5%

Elemental analysis for C₂₁H₂₈FN₃O.½H₂O

Calcd. (%): C, 68.82; H, 7.98; N, 11.47.

Found. (%): C, 68.86; H, 7.98; N, 11.42.

¹H NMR (200 MHz): δ7.95-8.05(m, 2H), 7.09-7.21(m, 2H), 6.83(s, 1H), 4.38(t,J=13.2 Hz,2H), 2.65(s,3H), 2.27-2.38(Complex m,6H), 1.74-1.88 (m,2H), 1.43-1.61 (Complex m,10H)

### Preparation Example 5

### NS-7

Eighteen kg of 4-(4-fluorophenyl)-2-methyl-6-(5-piperidinopentyloxy)pyrimidine hemihydrate obtained in the Preparation Example 4 was dissolved in 142.2 kg of acetone, and thereto was added 5.0 kg of hydrochloric acid. The mixture was stirred at about 20° C. for 1 hour. The precipitated crystals were isolated, washed with acetone and dried to provide 17.4 kg of the title compound as crude crystals. Seventeen kg of this crude crystal was recrystallized from ethanol to provide 15.4 kg of the title compound as white crystals (yield: 91%).

### Formulation Example 1

### Injections

According to the following recipe, an injection, 1 ml, can be prepared in a conventional method.

Recipe: NS-7, 1 mg; sodium chloride, 9 mg; sterile water for injection, q.s.

### Formulation Example 2

### Injections

According to the following recipe, an injection, 1 ml, can be prepared in a conventional method.

Recipe: NS-7, 1 mg; glucose, 48 mg; sodium dihydrogenphosphate, 1.25 mg; disodium hydrogenphosphate, 0.18 mg; sterile water for injection, q.s.

### Formulation Example 3

### Injections

According to the following recipe, injections, 1 ml, can be prepared in a conventional method.

Recipe: NS-7, 1 mg; sorbit, 48 mg; benzyl alcohol, 20 mg; sodium dihydrogenphosphate, 2.5 mg; disodium hydrogenphosphate, 0.36 mg; sterile water for injection, q.s.

### Formulation Example 4

### Tablets

According to the following recipe, a tablet, 120 mg, can be prepared in a conventional method.

Recipe: NS-7, 3 mg; lactose, 58 mg; corn starch, 30 mg; crystalline cellulose, 20 mg; hydroxypropylcellulose, 7 mg; magnesium stearate, 2 mg.

### Formulation Example 5

### Drip Infusions

According to the following recipe, a drip infusion in bag, 500 ml, can be prepared in a conventional method.

Recipe: NS-7, 25 mg; sodium chloride, 4.5 g; sterile water for injection, q.s.

### Formulation Example 6

### Sustained Infusions

According to the following recipe, pre-filled syringe kit formulation, 20 ml, can be prepared in a conventional method.

Recipe: NS-7, 20 mg; sodium chloride, 180 mg; sterile water for injection, q.s.

### Experimental Example 1

### Inhibition of Neurologic Injury after Spinal Cord Ischemia in Rabbit

1. Experimental Method

(1) Preparation of Rabbit Model of Spinal Cord Ischemia

Twenty-six male New Zealand White rabbits weighting 2.2 to 3.0 kg were used in this experiment. Spinal cord ischemia was induced by occluding the abdominal aorta with snares for 20 minutes just distal to the renal arteries.

Surgical preparation was conducted according to the method described in the literature, Terada H. et. al., J. Thorac. Cardiovasc. Surg. 2001; 122: 979-85. A 24-gauge venous catheter was placed in the marginal ear vein for drug administration. The rabbits were anesthetized with intravenous sodium pentobarbital (25 mg/kg) and allowed to breathe spontaneously. Lidocaine (0.5%) was administered at the site of the skin incision as local anesthesia. The left common carotid artery was cannulated with a 24-gauge catheter for monitoring the arterial pressure. Core body temperature was continuously monitored with a rectal probe and was maintained at 38.5° C. with the aid of heating lamp. The infrarenal abdominal aorta was exposed through a transperitoneal approach. After systemic heparinization (200 U/kg), spinal cord ischemia was induced by occluding the abdominal aorta with snares for 20 minutes just distal to the renal arteries and just above the aortic bifurcation. Then, the snares were released, and the flank was closed.

(2) Drug Administration

NS-7 prepared in Preparation Example 2, which is also available from Nippon Shinyaku Co., Ltd, Japan, was dissolved in 0.9% saline to yield a 2.5 mg/ml solution. The rabbits were assigned randomly to 1 of the 4 groups and subjected to the treatment of (1) above in the following manner. The sham group (n=3) underwent the same operation without aortic occlusion. The control group (n=7) received saline (1 ml) intravenously 15 minutes before aortic occlusion. NS-7 (1 mg/kg) was administered intravenously as a bolus dose 15 minutes before aortic occlusion in group A (n=8) or just after releasing the snares (at the onset of reperfusion) in group B (n=8).

(3) Tissue Extraction

All animals were killed by means of a lethal injection of pentobarbital (200 mg/kg) 48 hours after operation. Spinal cords were quickly harvested and sectioned from the low thoracic level to the low lumbar level into the following 4 segments: low thoracic (LT=T10-T12), upper lumbar (UL=L1-L2), mid lumbar (ML=L3-L4), and low lumbar (LL=L5-L6).

(4) Statistical Analysis

The data were presented as means±SD. Statistical analysis of neurologic scores and histologic scores were performed with Mann-Whitney U test. The physiologic data were processed by means of analysis of variance for reported measures. The numbers of TUNEL-positive neurons and the infarct size of spinal cords were evaluated by 1-way analysis of variance, followed by the Dunnett test when significant differences were identified.

2. Results

(1) Physiologic Parameters

All the animals survived the entire observation period. Body weight, rectal temperature, and mean arterial blood pressure values at baseline, 10 minutes after ischemia, and 10 minutes after reperfusion are shown in Table 1. There were no significant differences in the weights of the animals. Rectal temperature and mean arterial blood pressure were not significantly different among the 4 groups at each time point.

TABLE 1
Physiologic Parameters

| | Sham | Control | Group A | Group B |
|---|---|---|---|---|
| | (n = 3) | (n = 7) | (n = 8) | (n = 8) |
| Weight (kg) | 2.4 ± 0.1 | 2.3 ± 0.1 | 2.6 ± 0.2 | 2.5 ± 0.2 |
| Rectal Temp. (° C.) | | | | |
| Baseline | 38.7 ± 0.6 | 38.7 ± 0.4 | 38.4 ± 0.7 | 38.5 ± 0.4 |
| Ischemia, 10 min | | 38.3 ± 0.5 | 38.1 ± 0.4 | 38.4 ± 0.5 |
| Reperfusion, 10 min | | 38.5 ± 0.3 | 38.1 ± 0.2 | 38.6 ± 0.5 |
| MAP (mmHg) | | | | |
| Baseline | 83 ± 8 | 86 ± 5 | 83 ± 5 | 91 ± 6 |
| Ischemia, 10 min | | 82 ± 3 | 85 ± 5 | 86 ± 7 |
| Reperfusion, 10 min | | 79 ± 12 | 78 ± 10 | 81 ± 8 |

MAP: mean arterial blood pressure

Values are expressed as means±SD. There were no significant differences in these physiologic parameters among the 4 groups at any time point. The above results show that the pharmaceutical composition of the present invention is free from cardiovascular side-effects which conventional Na⁺/Ca⁺ channel blockers have, and is safe.

(2) Neurologic Assessment

Hind-limb motor function was scored 24 and 48 hours after the operation by the modified Tarlov scale. The neurologic function was graded by an observer without knowledge of the treatment. 0, No movement; 1, slight movement; 2, sit with assistance; 3, sit alone; 4, weak hop; 5, normal hop.

The results are shown in Table 2. All the rabbits in the sham group remained normal (Tarlov score of 5) throughout the observation period. Twenty minutes of infrarenal aortic occlusion resulted in severe lower extremity neurologic deficits in the control group. NS-7 treatment (group A and B) remarkably enhanced the recovery of motor function in the hind limbs both 24 hours and 48 hours after the operation (P<0.01 for group A and B compared with the control group at both time points). Compared with group A, better Tarlov scores were obtained with animals that received NS-7 at the onset of reperfusion (group B), but the difference was not significant.

TABLE 2
Neurologic score at 24 and 48 hours after the operation

| | Sham | | Control G | | Group A | | Group B | |
|---|---|---|---|---|---|---|---|---|
| Tarlov | (n = 3) | | (n = 7) | | (n = 8) | | (n = 8) | |
| score | 24 h | 48 h | 24 h | 48 h | 24 h* | 48 h^{#} | 24 h* | 48 h^{#} |
| 0 | 0 | 0 | 5 | 6 | 0 | 0 | 0 | 0 |
| 1 | 0 | 0 | 0 | 0 | 0 | 0 | 0 | 0 |
| 2 | 0 | 0 | 0 | 0 | 0 | 1 | 0 | 1 |
| 3 | 0 | 0 | 1 | 0 | 2 | 2 | 2 | 2 |
| 4 | 0 | 0 | 1 | 1 | 3 | 4 | 1 | 1 |
| 5 | 3 | 3 | 0 | 0 | 3 | 1 | 5 | 4 |

*P < 0.01 compared with the control group of 24 hours (Mann-Whitney U test).
^{#}P < 0.01 compared with the control group of 48 hours (Mann-Whitney U test).

(3) Observation of Infarct Size of Spinal Cords by TTC Staining

To measure the infarct size of spinal cords, 2,3,5-triphenyltetrazonlium chloride (TTC; Sigma Chemical, St Louis, Mo.) staining was used, which can turn the viable tissue brick red and the necrotic tissue pale. Spinal cords (mid lumbar segment) were quickly removed, and transverse sections of about 2 mm in thickness were cut. Slices were then incubated for 30 minutes in 1% TTC at 37° C. After fixation in a 10% formalin solution, the TTC-stained sections were photographed.

Photographs of TTC staining of the spinal cords (mid lumbar level) are shown in FIG. 1. In FIG. 1, viable tissue is brick red, whereas infarcted tissue is pale. White matter is also pale. The specimen from the sham group (A) shows no infarction in the gray matter, whereas almost the entire anterior horns show infarction in the section from the control group (B). Minimal gray matter infarction is shown in rabbits that received NS-7 before ischemia (C) or at the onset of reperfusion (D) (respectively the group A or B).

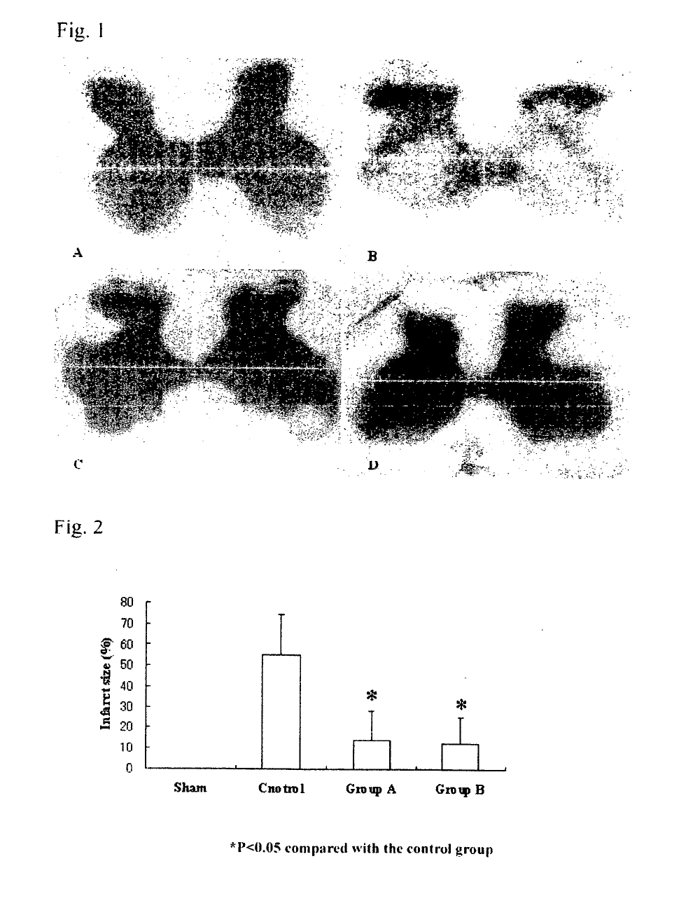

The area of infarction was calculated with National Institutes of Health Image software. The infarct size was expressed as a percentage of the area of the whole gray matter. The results are shown in FIG. 2. The infarct size (%) is plotted on the vertical axis and therein are shown the results of sham, control, Group A and Group B starting from the left. *P<0.05 compared with the control group.

Forty-eight hours after the spinal cord ischemia, the average size of the infarction in the control group was 54.7±19.8%. Administration of NS-7 was effective in reducing the infarct size when it was injected before ischemia (14.0±14.3%, P<0.05, compared with the control group) or at the onset of reperfusion (12.1±12.8%, P<0.05).

As shown above, the results of TTC staining and the average neurologic scores at 48 hours are in good correlation.

(4) Histopathologic Examination

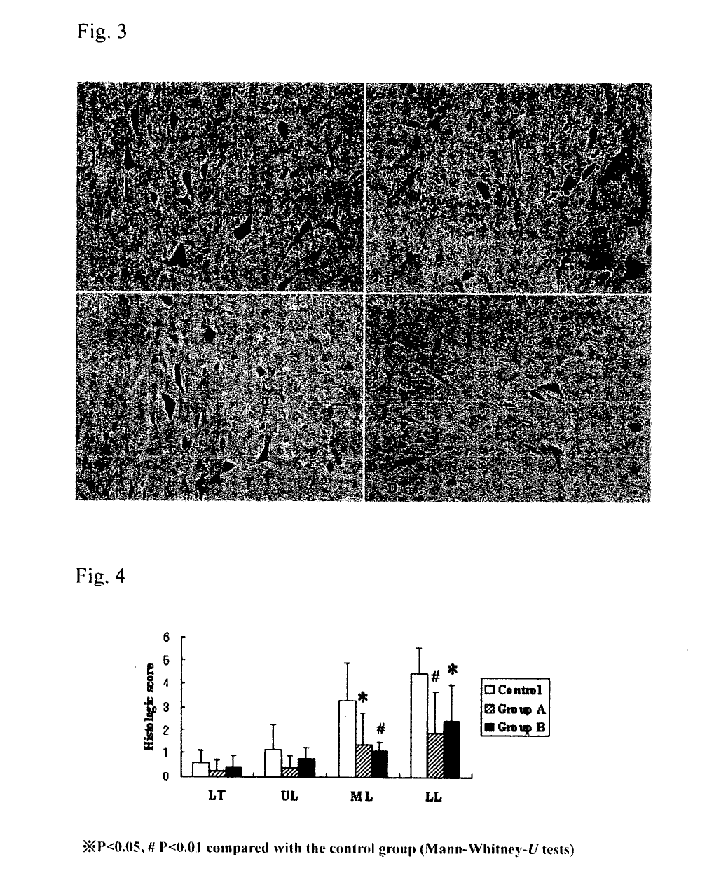

The spinal cords were fixed in 10% formalin and embedded in paraffin. Two transverse sections (4 μm) of each segment were cut, stained with hematoxylin and eosin (HE) and photographed. The results are shown in FIG. 3 (mid lumbar) (original magnification 200×). As shown in FIG. 3, no histologic changes are shown in the ventral horns of a sham-operated rabbit (A), and the specimen from a control animal (B) exhibits eosinophilic neuronal degeneration (arrows), vacuolization, and necrosis. On the other hand, animals that received NS-7 before ischemia (C) or at the onset of reperfusion (D) (respectively, group A or B) show minimal evidence of cellular damage.

An investigator who was unaware of the animal group and neurologic outcome examined each slide.

Histologic damage was graded by using semiquantitative scoring method (Table 3) (Martelli E. et. al., J. Vasc. Surg. 2002; 35: 547-53).

TABLE 3
Semiquantitative scoring for histologic assessment
of spinal cord injury (invasion)

| Histologic assessment | Score |
|---|---|
| Healthy | 0 |
| Perineural edema or scattered 1-cell necrosis | 1 |
| Unilateral necrosis of central medial portion of anterior horn | 2 |
| Bilateral necrosis of central medial portion of anterior horn | 3 |
| Unilateral necrosis of entire anterior horn | 4 |
| Bilateral necrosis of entire anterior horn | 5 |

The graded results are shown in FIG. 4. Mean histologic scores at different sites of the spinal cord are plotted on the vertical axis, and therein are shown the results of LT, Low thoracic; UL, upper lumbar; ML, mid lumbar; and LL, low lumbar starting from the left. *P<0.05, #P<0.01 compared with the control group (Mann-Whitney U tests).

As shown in FIG. 4, no sign of spinal cord damage was observed in the hematoxylin and eosin-stained sections at any level in the sham operated rabbits. The low thoracic and upper lumbar regions of the spinal cords from the other 3 groups showed no or only minimal injury. Although minor histologic changes occurred more frequently in the control group, the differences in histologic scores were not statistically significant. Examination of the mid lumbar and low lumbar spinal cords of the control group revealed severe neuronal damage, as evidenced by eosinophilic neuronal degeneration, vacuolization, and necrosis (FIG. 3, B). However, the rabbits treated with NS-7 15 minutes before aortic occlusion (group A) or at the onset of reperfusion (group B) showed only slight changes in the spinal cords at these 2 levels, and the average histologic scores were significantly lower than those of the control group (FIG. 4).

(5) TUNEL Staining

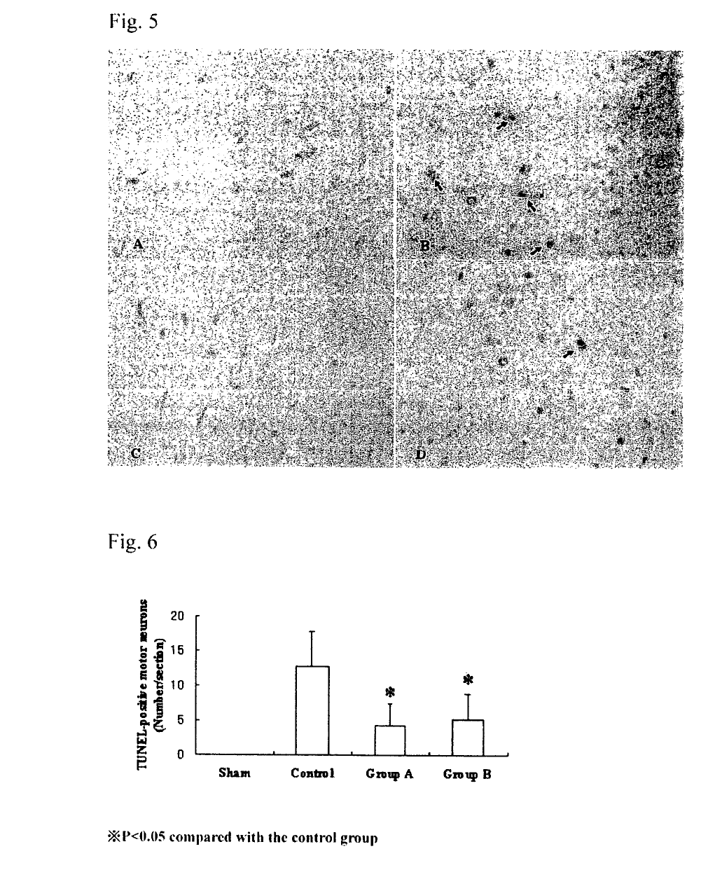

Paraffin-embedded sections (mid lumbar segment) were used for in situ terminal deoxynucleotidyl transferase (TDT)-mediated dUTP-biotin nick end labeling (TUNEL staining) with Apop Tag (Intergen Co, New York, N.Y.), according to the protocols recommended by the manufacturer. In brief, after being deparaffinized, the sections were treated with proteinase and 0.3% H₂O₂. Then the sections were incubated for 60 minutes with terminal deoxynucleotidyl transferase enzyme at 37° C. The color was developed with a DAB/H₂O₂ solution. The sections were counterstained in methyl green and photographed. The results are shown in FIG. 5 (original magnification 200×). In FIG. 5, A represents sham-operated group; B, control group; C, treated group that received NS-7 before ischemia; and D, treated group that received NS-7 at the onset of reperfusion. Arrows exhibits TUNEL-positive motor neuron.

The number of motor neurons that were positive in TUNEL staining was counted in 3 sections, and the values were averaged (Sakurai M., J. Thorac. Cardiovasc. Surg. 2000; 120: 1148-57, Lang-Lazdunski L., J. Vasc. Surg. 2003; 38: 564-75). The results are shown in FIG. 6. In FIG. 6, mean value of TUNEL-positive motor neurons of each slice is plotted on the vertical axis, and therein are shown the results of Sham, Control, Group A and Group B starting from the left. *P<0.05 compared with the control group.

Cells with double-strand breaks in DNA, which are suggestive of apoptosis, can be detected by means of TUNEL staining. The spinal cords from the rabbits in the sham group showed no evidence of TUNEL staining. Samples from the control group stained strongly positive, with numerous TUNEL positive motor neurons scattered in the ventral gray matter (FIG. 5, B). NS-7 injected 15 minutes before ischemia or at the onset of reperfusion strongly reduced the total number of apoptotic motor neurons compared with that seen in the control group (P<0.05, compared with the control, FIG. 6). No significant difference in the total number of apoptotic motor neurons was found between the two NS-7-treated groups.

The experimental results above demonstrate that the pharmaceutical composition of the present invention has protective effect against both of neuronal necrosis and apoptosis at the time of spinal cord ischemia and excellent preventive and restorative effects on neuronal injury. Furthermore, the above results show that the pharmaceutical composition of the present invention can be administered at any time before and after interruption of blood flow until the onset of reperfusion.

### INDUSTRIAL APPLICABILITY

The active ingredient [1] of the pharmaceutical composition of the present invention has protective effects on spinal cord neurons at the time of spinal cord ischemia. Therefore, the pharmaceutical composition of the present invention can be used for preventing and/or treating the neuronal damage due to spinal cord injury.

1. A method for preventing or treating neurologic damage after spinal cord injury in a mammal including human in need thereof which comprises administering an effective amount of a compound of the following general formula [I]: 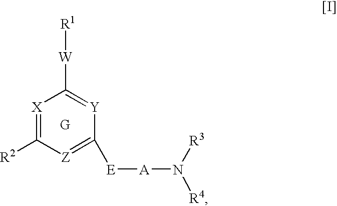
or a salt thereof, or a solvate thereof;
in the formula [I], R¹ represents an aryl group that may be substituted or a 5- through 10-membered heteroaromatic group that may be substituted, wherein the heteroaromatic group may be a monocyclic or fused ring system containing at least one heteroatom selected from the group consisting of nitrogen, oxygen, and sulfur as a ring member; additionally, the aryl and heteroaromatic group each may be substituted with one to three substituents which are the same or different and selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro;
R² represents hydrogen, alkyl, alkenyl, cycloalkyl, cycloalkylalkyl, hydroxyalkyl, haloalkyl, alkoxy, alkylthio, amino, monoalkylamino, dialkylamino, or phenyl. The phenyl may be substituted with one to three substituents which are the same or different and selected from the group consisting of halogen, alkyl, and alkoxy;
R³ and R⁴ may be the same or different and each represents hydrogen or alkyl that may be substituted by hydroxy, alkoxy, amino, monoalkylamino, or dialkylamino, or R³ and R⁴ taken together with the adjacent N atom may represent a 4- through 8-membered cyclic amino group of the formula NR³R⁴; the cyclic amino group may have N, O, or S in addition to said N atom as a ring member and be substituted with one to three substituents that are the same or different and selected from the group consisting of alkyl, alkoxy, hydroxy, oxo, amino, monoalkylamino, dialkylamino, aryl that may be substituted, and pyridyl that may be substituted;
R³ and R⁴ taken together with the adjacent N atom may form an oxide;
A represents alkylene of 2-10 carbon atoms which may be substituted by one or more substituents which may be the same or different and selected from the group consisting of alkoxy, hydroxy, and oxo in optional substitutable positions;
E represents O or S;
W represents a single bond, O, S, or (CH₂)ₙ, wherein CH₂ may be substituted with alkyl and n is an integer of 1 or 2;
X, Y, and Z may be the same or different and each represents CH, CR or N, wherein R represents alkyl, with the proviso that the case in which X, Y, and Z concurrently represent CH or CR wherein R represents alkyl is excluded;
ring G represents pyridine, pyrimidine, or 1,3,5-triazine; and
when one to three of X, Y, and Z represent N, one of them may form an oxide.

2. The method according to claim 1, wherein R¹ represents halogen-substituted phenyl; R² represents alkyl or haloalkyl; R³ and R⁴ taken together with the adjacent N atom represent a 4-membered cyclic amino group containing only one nitrogen atom as the ring-constituent heteroatom of the formula —NR³R⁴; A represents alkylene of 3-6 carbon atoms; E represents O or S; W represents a single bond; X and Z each represent N with Y representing CH, or Z represents N with X and Y respectively representing CH.

3. The method according to claim 1, wherein NR³R⁴ represents piperidino; A represents alkylene of 4-6 carbon atoms; E represents O; W represents a single bond; and X and Z each represent N with Y representing CH or Z represents N with X and Y each representing CH.

4. The method according to claim 1, wherein the compound is selected from the group consisting of:
4-(4-fluorophenyl)-2-methyl-6-(4-piperidinobutoxy)pyrimidine,
4-(4-fluorophenyl)-2-methyl-6-(5-piperidinopentyloxy)pyrimidine,
4-(4-fluorophenyl)-2-methyl-6-(6-piperidinohexyloxy)pyrimidine,
4-(4-fluorophenyl)-2-methyl-6-(1-methyl-4-piperidinobutoxy)pyrimidine,
2-(4-fluorophenyl)-4-methyl-6-(4-piperidinobutoxy)pyrimidine,
4-(4-fluorophenyl)-2-methyl-6-(3-piperidinopropoxy)pyridine, and
4-(4-fluorophenyl)-2-methyl-6-(5-piperidinopentyloxy)pyridine,
or a salt thereof, or a solvate thereof.

5. A method for protecting spinal cord neuronal cells against spinal cord ischemia in a mammal including human in need thereof which comprises administering an effective amount of a compound of the following general formula [I]: 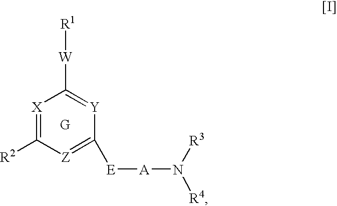
or a salt thereof, or a solvate thereof;
in the formula [I], R¹ represents an aryl group that may be substituted or a 5- through 10-membered heteroaromatic group that may be substituted, wherein the heteroaromatic group may be a monocyclic or fused ring system containing at least one heteroatom selected from the group consisting of nitrogen, oxygen, and sulfur as a ring member; additionally, the aryl and heteroaromatic group each may be substituted with one to three substituents which are the same or different and selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro;
R² represents hydrogen, alkyl, alkenyl, cycloalkyl, cycloalkylalkyl, hydroxyalkyl, haloalkyl, alkoxy, alkylthio, amino, monoalkylamino, dialkylamino, or phenyl. The phenyl may be substituted with one to three substituents which are the same or different and selected from the group consisting of halogen, alkyl, and alkoxy;
R³ and R⁴ may be the same or different and each represents hydrogen or alkyl that may be substituted by one or two substituents which are the same or different and selected from the group consisting of hydroxy, alkoxy, amino, monoalkylamino, and dialkylamino, or R³ and R⁴ taken together with the adjacent N atom may represent a 4- through 8-membered cyclic amino group of the formula NR³R⁴; the cyclic amino group may have N, O, or S in addition to said N atom as a ring member and be substituted with one to three substituents that are the same or different and selected from the group consisting of alkyl, alkoxy, hydroxy, oxo, amino, monoalkylamino, dialkylamino, aryl that may be substituted, and pyridyl that may be substituted;
R³ and R⁴ taken together with the adjacent N atom may form an oxide;
A represents alkylene of 2-10 carbon atoms which may be substituted by alkoxy, hydroxy, or oxo in optional substitutable positions;
E represents O or S;
W represents a single bond, O, S, or (CH₂)ₙ, wherein CH₂ may be substituted with alkyl and n is an integer of 1 or 2;
X, Y, and Z may be the same or different and each represents CH, CR or N, wherein R represents alkyl, with the proviso that the case in which X, Y, and Z concurrently represent CH or CR wherein R represents alkyl is excluded;
ring G represents pyridine, pyrimidine, or 1,3,5-triazine; and
when one to three of X, Y, and Z represent N, one of them may form an oxide.

6. The method according to claim 5, wherein R¹ represents halogen-substituted phenyl; R² represents alkyl or haloalkyl; R³ and R⁴ taken together with the adjacent N atom represent a 4-membered cyclic amino group containing only one nitrogen atom as the ring-constituent heteroatom of the formula —NR³R⁴; A represents alkylene of 3-6 carbon atoms; E represents O or S; W represents a single bond; X and Z each represent N with Y representing CH, or Z represents N with X and Y each representing CH.

7. The method according to claim 5, wherein NR³R⁴ represents piperidino; A represents alkylene of 4-6 carbon atoms; E represents O; W represents a single bond; and X and Z each represent N with Y representing CH or Z represents N with X and Y each representing CH.

8. The method according to claim 5, wherein the compound is selected from the group consisting of:
4-(4-fluorophenyl)-2-methyl-6-(4-piperidinobutoxy)pyrimidine,
4-(4-fluorophenyl)-2-methyl-6-(5-piperidinopentyloxy)pyrimidine,
4-(4-fluorophenyl)-2-methyl-6-(6-piperidinohexyloxy)pyrimidine,
4-(4-fluorophenyl)-2-methyl-6-(1-methyl-4-piperidinobutoxy)pyrimidine,
2-(4-fluorophenyl)-4-methyl-6-(4-piperidinobutoxy)pyrimidine,
4-(4-fluorophenyl)-2-methyl-6-(3-piperidinopropoxy)pyridine, and
4-(4-fluorophenyl)-2-methyl-6-(5-piperidinopentyloxy)pyridine,
or a salt thereof, or a solvate thereof.

9. Use of a compound of the general formula [I]: 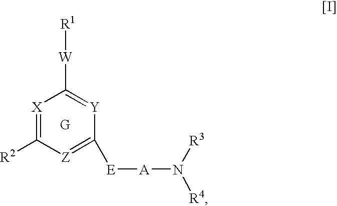
or a salt thereof, or a solvate thereof, for the manufacture of a medicament for the prevention or the treatment of neurologic damage after spinal cord injury;
in the formula [I], R¹ represents an aryl group that may be substituted or a 5- through 10-membered heteroaromatic group that may be substituted, wherein the heteroaromatic group may be a monocyclic or fused ring system containing at least one heteroatom selected from the group consisting of nitrogen, oxygen, and sulfur as a ring member; additionally, the aryl and heteroaromatic group each may be substituted with one to three substituents which are the same or different and selected from the group consisting of hydroxy, halogen, alkyl, haloalkyl, hydroxyalkyl, aralkyl, alkenyl, alkoxy, haloalkyloxy, alkylthio, cycloalkyl, cycloalkylalkyl, cycloalkyloxy, alkylsulfonyl, sulfamoyl, alkanoyl, amino, monoalkylamino, dialkylamino, carboxy, alkoxycarbonyl, cyano, and nitro;
R² represents hydrogen, alkyl, alkenyl, cycloalkyl, cycloalkylalkyl, hydroxyalkyl, haloalkyl, alkoxy, alkylthio, amino, monoalkylamino, dialkylamino, or phenyl. The phenyl may be substituted with one to three substituents which are the same or different and selected from the group consisting of halogen, alkyl, and alkoxy;
R³ and R⁴ may be the same or different and each represents hydrogen or alkyl that may be substituted by one or two substituents which are the same or different and selected from the group consisting of hydroxy, alkoxy, amino, monoalkylamino, and dialkylamino, or R³ and R⁴ taken together with the adjacent N atom may represent a 4-through 8-membered cyclic amino group of the formula NR³R⁴; the cyclic amino group may have N, O, or S in addition to said N atom as a ring member and be substituted with one to three substituents that are the same or different and selected from the group consisting of alkyl, alkoxy, hydroxy, oxo, amino, monoalkylamino, dialkylamino, aryl that may be substituted, and pyridyl that may be substituted;
R³ and R⁴ taken together with the adjacent N atom may form an oxide;
A represents alkylene of 2-10 carbon atoms which may be substituted by alkoxy, hydroxy, or oxo in optional substitutable positions;
E represents O or S;
W represents a single bond, O, S, or (CH₂)ₙ, wherein CH₂ may be substituted with alkyl and n is an integer of 1 or 2;
X, Y, and Z may be the same or different and each represents CH, CR or N, wherein R represents alkyl, with the proviso that the case in which X, Y, and Z concurrently represent CH or CR wherein R represents alkyl is excluded;
ring G represents pyridine, pyrimidine, or 1,3,5-triazine; and
when one to three of X, Y, and Z represent N, one of them may form an oxide.
